(19) United States
(12) Patent Application Publication
(10) Pub. No.: US 2014/0308231 A1
(43) Pub. Date: Oct. 16, 2014

(54) AUTOTROPH-BASED DEODORIZER AND METHOD FOR MANUFACTURING THE SAME

(71) Applicant: Song BYUNG JOON, Paju-si (KR)

(72) Inventor: Song BYUNG JOON, Paju-si (KR)

(21) Appl. No.: 14/106,009

(22) Filed: Dec. 13, 2013

(30) Foreign Application Priority Data
Apr. 12, 2013 (KR) 10-2013-0040386

(51) Int. Cl. A61L 9/01

(52) U.S. Cl. CPC A61L 9/01
USPC 424/76.1; 435/34

(57) ABSTRACT

Disclosed are a deodorizer using an autotrophic microbe, and a method for preparing the same. The deodorizer is prepared through a culturing step for amplifying an autotrophic microbe of interest, a sterilizing and purifying step for removing impurities and harmful microbes other than the autotrophic microbe, and a harvesting step for collecting the filtered autotrophic microbes. The microbial deodorizes is effective for removing odorous emissions attributed to ammonia, trimethylamine, formaldehyde, and hydrogen sulfide and is safe to the body and the environment including air and water.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

REFERENCE TO AN APPENDIX SUBMITTED ON COMPACT DISC

Not applicable.

### BACKGROUND OF THE INVENTION

1. Field of the Invention

The present invention relates to a deodorizer using an autotrophic microbe. More particularly, the present invention relates to a microbial deodorizer which is effective for removing odorous emissions attributed to ammonia, trimethylamine, formaldehyde, and hydrogen sulfide and which is safe to the body and the environment including air and water because it undergoes a sterilization and purification step when boiled at 99° C. for 5 min.

2. Description of Related Art Including Information Disclosed Under 37 CFR 1.97 and 147 CFR 1.98.

Foul odor from living environments, such as domestic sewage, excreta, furniture, facilities, constructs, pets, etc. is generally accounted for by nitrogen compounds, sulfides, aldehydes, and other carbon compounds. Representative among the causes of bad odor are ammonia, trimethylamine, formaldehyde, hydrogen sulfide, and hydrocarbons. Typically, air purifiers, chemical deodorizers, and microbial deodorizers are used to remove foul odors.

Due to their artificial and synthetic chemicals, chemical deodorants however, emit ingredients detrimental to the body, causing the production of air pollutants. That is, chemical deodorizers, although effective for deodorization, destroy the virtuous cycle of the natural ecosystem in a long-term aspect.

Microbial deodorizers exhibit rather adverse effects because intensive sterilization for eliminating contaminants or putrefactive microbes, which may be introduced or generated during manufacturing processes, cannot be carried out lest the main microbes would be killed.

For example, Korean Patent No. 10-1114185 discloses a deodorant composition comprising a fermentation product fermented from fermentation materials including a rice bran extract, a sugar and solar salt by an effective microorganism (EM), plus the plant surfactant cocobetaine and a hot water extract of rosemary, and a manufacturing method thereof.

Because it is based on a heterotrophic microorganism, this deodorant composition cannot be subjected to a sterilization process for removing detrimental impurities or microbes other than the effective microorganism. Thus, it is not recommended to elicit the deodorization effect by applying the deodorant composition directly to living bodies, such as humans, animals or plants.

Korean Patent. No. 10-0958064 addresses a microbial agent for wastewater purification and a purifying device comprising the same, which are configured to improve both foul odor and water quality, with the aim of solving problems with organic wastewater disposal such as high expense and low efficiency, and coping with strengthened environmental regulations.

This microbial agent is produced without any sterilization or purification process for removing detrimental microbes or impurities other than working microbes, so that it is not recommended to be directly sprayed to air, surroundings, or human or animal bodies.

Keeping in mind the above problems encountered in the prior art, the present invention has been made by selectively utilizing autotrophic microbes that are believed to purify the atmosphere of the proto-earth to establish an environmental background for the appearance of higher organisms, such as humans, animals and plants, that is, by culturing the autotrophic microbes, and performing sterilization and purification to remove impurities and microorganisms other than the microbes of interest to afford an environment-friendly microbial deodorizer product which can effectively remove odorous emissions from household sources in sympathy with the virtuous cycle of the ecosystem and which is harmless to the body and the environment.

### PRIOR ART DOCUMENTS

### Patent Documents

1. Korean Patent No. 10-114185

2. Korean Patent No. 10-0958064

### BRIEF SUMMARY OF THE INVENTION

It is therefore an object of the present invention to provide an environment-friendly microbial deodorizer using an autotrophic microbe which can effectively remove various odorous emissions from living environment sources, in harmony with the virtuous cycle of the ecosystem without harm to the body and the environment.

It is another object of the present invention to provide a process for manufacturing the environment-friendly microbial deodorizer.

To achieve the above objects, the microbial deodorizer using an autotrophic microbe in accordance with the present invention is manufactured by a process comprising a screening step (S1), a culturing (S2-1) and mixing (S2-2) step, a sterilizing step (S3), a purifying step (S4), and a harvesting step (S5).

In the screening step (S1), a purple sulfur bacterial strain, found in soil, is selected from the group consisting of Allochromaium palmeri, Ectothiorhodosinus mongolicus, Halochromatium roseum, and a combination thereof.

The second step may be divided into sub-steps of culturing (S2-1) and mixing (S2-2). The autotrophic microbial strain selected in the screening step is cultured in a medium, adjusted to a pH of 6.0-7.0, containing potassium phosphate, magnesium sulfate, sodium chloride, ammonium chloride, and calcium chloride in an incubator at 43-45° C. for 190-210 hrs under the light of 5000 Lux or higher (S2-1), the autotrophic bacteria amplified above are adjusted to a density of 4-5×10⁵ cfu/ml with, as needed, a distilled water (S2-2).

Next, the sterilizing step (S3) is carried out by boiling the diluted culture of the autotrophic bacteria at 99° C. for 5 min so as to remove other bacteria and contaminants which could be introduced during the above steps.

After the sterilization, the microbial culture is cooled to room temperature in a clean room and incubated for 48-60 hrs without feeding an additional medium thereto, followed by filtration (purifying step S4).

Finally, the autotrophic microbe filtrate is maintained at a density of 4-5×10⁵ cfu/ml and processed into a commercial product (harvesting and commercializing step (S5).

An examination was made of the ability of the microbial deodorizer of the present invention to deodorize ammonia, trimethylamine, formamide, and hydrogen sulfur by the Korea Conformity Laboratories.

The microbial deodorizer of the present invention was found to have faster and improved deodorization, compared to conventional microbial agents prepared without sterilization and purification, as demonstrated by the testing of the authorized office (Tables 1-8).

As described hereinbefore, the present invention is characterized by the sterilization and purification of microbial agents, for example, those of Korea Patent No. 10-0958064, in order to make them harmless to the body and the environment without degrading the deodorizing ability. In a fish bowl filled with the microbial deodorizer of the present invention, gold fish were safely grown for three months (FIG. 3). In addition, the microbial deodorizer of the present invention was found to be safe to the body and the environment because of no detection of heavy metals and other harmful substances therefrom, as described in a test report prepared by the Korea Conformity Laboratories (Table 9).

### BRIEF DESCRIPTION OF THE SEVERAL VIEWS OF THE DRAWINGS

The above and other objects, features and advantages of the present invention will be more clearly understood from the following detailed description taken in conjunction with the accompanying drawings, in which:

FIG. 1 is a schematic view illustrating the degradation of odorous sources into non-odorous compounds by autotrophic microbes according to the present invention.

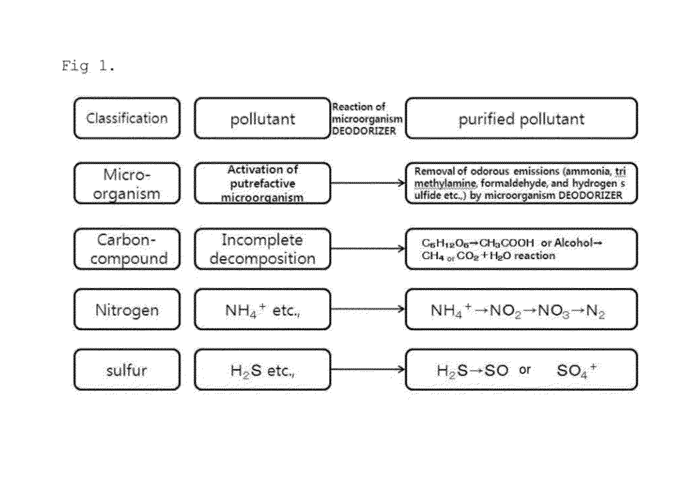

FIG. 2 is a flow chart, showing the manufacturing process of a microbial deodorizer of the present invention in which an autotrophic microbe is screened (S1), cultured and mixed (S2), sterilized (S3), purified (S4), and harvested (S5).

FIG. 3 is a photograph illustrating the safety of the microbial deodorizer which underwent the sterilizing (S3) and purifying step (S4), as demonstrated by the healthy growth of gold fish exposed thereto.

FIG. 4 is a picture taken of a commercial product of the autotrophic microbe according to the present invention.

FIG. 5 is a graph showing deodorizing effects on ammonia gas of two microbial agents which are prepared by conducting up to the culturing step (S2), and up to the sterilizing (S3) and purifying step (S4), respectively.

FIG. 6 is a graph showing deodorizing effects on trimethylamine gas of two microbial agents which are prepared by conducting up to the culturing step (S2), and up to the sterilizing (S3) and purifying step (S4), respectively.

FIG. 7 is a graph showing deodorizing effects on formaldehyde gas of two microbial agents which are prepared by conducting up to the culturing step (S2), and up to the sterilizing (S3) and purifying step (S4), respectively.

FIG. 8 is a graph showing deodorizing effects on hydrogen sulfide gas of two microbial agents which are prepared by conducting up to the culturing step (S2), and up to the sterilizing (S3) and purifying step (S4), respectively.

### DETAILED DESCRIPTION OF THE INVENTION

Below, a detailed description will be given of the present invention.

In accordance with an aspect thereof, the present invention addresses a microbial deodorizer.

For use in the microbial deodorizer of the present invention, a purple sulfur bacterial strain is selected from Allochromatium palmeri, Ectothiorhodosinus mongolicus, Halochromatium roseum, and a combination thereof. (Screening step S1).

That is, the three bacterial strains may be employed alone, or in combination in the microbial deodorizer of the present invention.

For use as a culture medium for the bacterial strains, potassium phosphate, magnesium sulfate, sodium chloride, ammonium chloride, and calcium chloride are mixed and dissolved in pure water and the solution is adjusted to a pH of 6.0-7.0. The bacterial strains are cultured in the medium in an incubator at 43-45° C. for 190-210 hrs under the light of 5000 Lux or higher until they are grown to a density of 4-5×10⁵ cfu/ml or greater (Culturing step S2-1 and mixing step S2-2).

Nitrogen, sulfur, aldehyde and hydrocarbon compounds, such as ammonia, trimethylamine, formaldehyde, hydrogen sulfide, etc. are predominantly responsible for odorous emissions from surrounding environment sources, such as household sewage, facilities, constructions, pets, etc.

Nitrogen, sulfur, and carbon compounds are fermented or degraded by the autotrophic microbes of the present invention, as exemplified as follows.

NH₄⁺→NO₂→NO₃→N2

H₂S→SO or SO₄³⁰

C₆H₁₂O₆→CH₃COOH or Alcohol→CH₄ or CO₂+H₂O

In addition to ammonia and hydrogen sulfide, there are other nitrogen and sulfur compounds that emit foul odors in living environments, examples include methylamine, ethylamine, dimethylamine, trimethylamine, isobutylamine, isoamylamin, phenylamine, putrescine, and cadaverine as nitrogen compounds, and methyl mercaptan, ethyl mercaptan (C₂H₃SH), dimethyl sulfide ((C₃)₂S), diethyl sulfide ((C₂H₃)₂S), and dimethyl disulfide (CH₃S═SCH₃) as sulfide compounds. These nitrogen and sulfur compounds are removed through the degradation mechanism above.

Among other carbon compounds serving as sources of odorous emissions are aldehydes and ketones (formalin, acetaldehyde, butylaldebyde, acrolein, acetone, acrylaldehyde), aliphatic acids (butyric acid, lactic acid, etc.), hydrocarbons (styrene, butyrene, etc.), chlorohydrocarbons (trichloroethylene, tetrachloroethylene, acrylic acid ester, acetic acid ester). These can be degraded in a similar mode of mechanism.

The break-down products concomitantly produced during the removal of odorous ingredients by the purple sulfur bacteria act to inhibit the growth of the putrefactive and pathogenic microorganisms activated by various pollution sources.

As well as functioning to deodorize odorous emissions from air and water, the microbial agent of the present invention is safe to the body and the environment, as manufactured through sterilization and purification steps for removing impurities and harmful microorganisms from the culture medium in which the autotrophic microbes of the present invention are cultured.

These effects of the present invention were evaluated as follows.

A conventional microbial agent prepared by the screening step (S1) and the culturing and mixing step (S2) was analyzed for deodorizing ability.

Separately, a microbial agent prepared by the method comprising the screening step (S1) to the purification step (S4) was subjected to a deodorization test, and a safety test for fish.

For deodorization testing of the microbial agents, a detection tube method was performed according to KS I2218 in the Korea Conformity Laboratories.

The microbial agent prepared by the culturing (S2-1) and mixing step (S2-2) were examined for deodorizing performance against ammonia, trimethylamine, formamide and hydrogen sulfide (Example 1).

The inventive microbial agent prepared by further conducting the sterilizing, step (S3) in which the microbe culture was boiled at 99° C. on a hot plate for 5 min and then cooled to room temperature, and the purifying step (S4) was also evaluated for ability to deodorize ammonia, trimethylamine, formamide, and hydrogen sulfide (Example 2).

As will be elucidated hereinafter, the microbial agent was found to deodorize the odorous emissions more fast with higher efficiency when prepared by conducting the screening step (S1) to the purifying step (S4) than the screening step (S1) and the culturing (S2-1) and mixing step (S2-2) only.

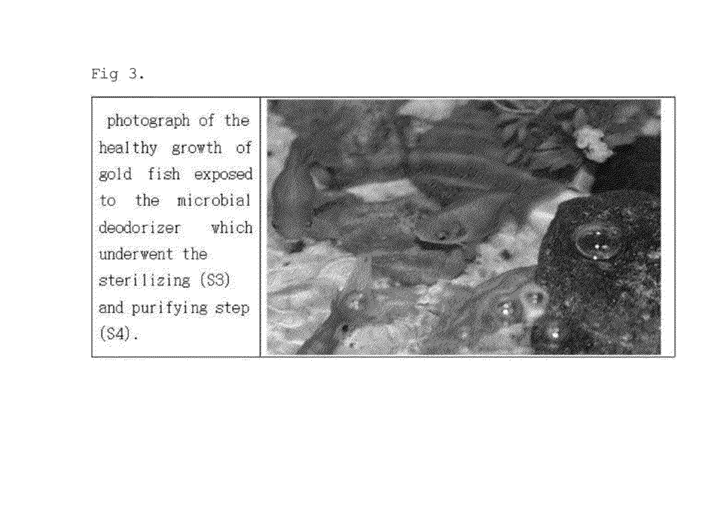

FIG. 3 shows fishes exposed to the microbe culture prepared following up to the sterilizing step (S3) and the purifying step (S4), demonstrating that the microbial deodorizer prepared by the method of the present invention is safe for use in living environments.

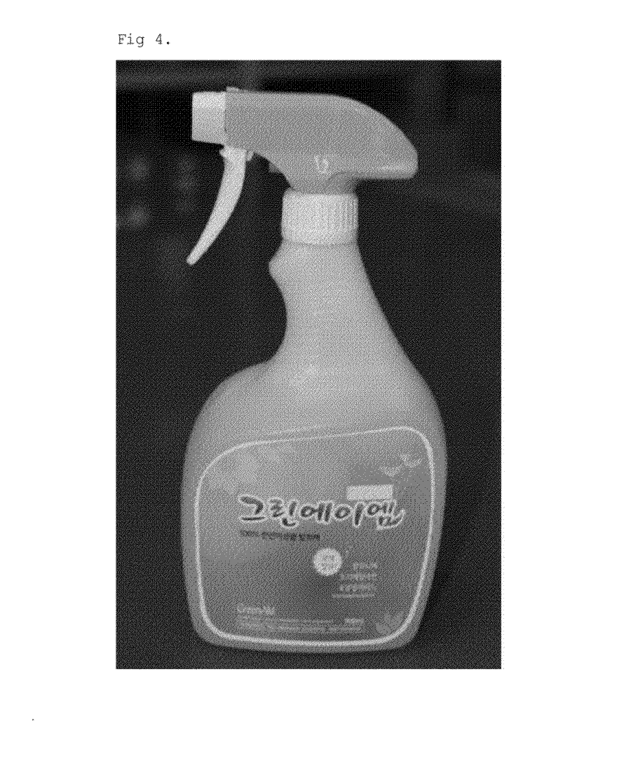

FIG. 4 is a picture of a commercial product containing the microbial deodorizer of the present invention.

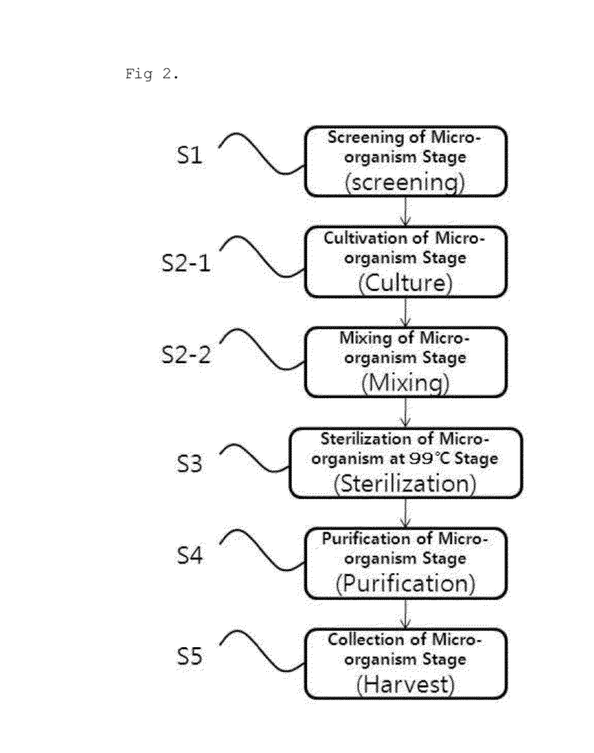

As delineated hitherto, the present invention provides a deodorizer comprising a microbial agent. The processes of manufacturing the deodorizer are illustrated in detail in FIG. 2, but are not limited to those shown in the flow chart.

A better understanding of the present invention may be obtained, through the following examples which are set forth to illustrate, but are not to be construed as limiting the present invention.

### EXAMPLE 1

A microbial agent prepared by conducting the screening step (S1) and the culturing (S2-1) and mixing (S2-2) step was measured for deodorizing performance against ammonia (Table 1), trimethylamine (Table 2), formaldehyde (Table 3), and hydrogen sulfide (Table 4).

TABLE 1
Deodorizing Effect of Microbial Agent Prepared by a Process Conducted
to the Culturing and Mixing Step (S2) on Ammonia Gas

| | Test Result | | |
|---|---|---|---|
| Test Item | Blank Conc. | Sample Conc. | Deodorization |
| Ammonia NH₃ | (μmol/mol) | (μmol/mol) | Rate (%) |
| 0 min | 50 | 50 | 0.0 |
| 30 min | 49 | 4 | 93.9 |
| 60 min | 49 | 2 | 95.9 |
| 90 min | 49 | 2 | 95.9 |
| 120 min | 48 | 1 | 97.9 |

TABLE 2
Deodorizing Effect of Microbial Agent Prepared by a Process Conducted
to the Culturing and Mixing Step (S2) on Triethylamine Gas

| Test Item | Test Result | | |
|---|---|---|---|
| Triethylamine | Blank Conc. | Sample Conc. | Deodorization |
| (CH₃)₃N | (μmol/mol) | (μmol/mol) | Rate (%) |
| 0 min | 50 | 50 | 0.0 |
| 30 min | 49 | 5 | 89.8 |
| 60 min | 49 | 4 | 91.8 |
| 90 min | 49 | 4 | 91.8 |
| 120 min | 48 | 3 | 93.8 |

TABLE 3
Deodorizing Effect of Microbial Agent Prepared by a Process Conducted
to the Culturing and Mixing Step (S2) on Formaldehyde Gas

| Test Item | Test Result | | |
|---|---|---|---|
| Formaldehyde | Blank Conc. | Sample Conc. | Deodorization |
| HCHO | (μmol/mol) | (μmol/mol) | Rate (%) |
| 0 min | 50 | 50 | 0.0 |
| 30 min | 49 | 10 | 79.6 |
| 60 min | 49 | 10 | 79.6 |
| 90 min | 49 | 10 | 79.6 |
| 120 min | 48 | 9 | 81.2 |

TABLE 4
Deodorizing Effect of Microbial Agent Prepared
by a Process Conducted to the Culturing and
Mixing Step (S2) on Hydrogen Sulfide Gas

| | |
|---|---|
| Test Item | Test Result |

| Hydrogen | Blank Conc. | Sample Conc. | Deodorization |
|---|---|---|---|
| Sulfide H₂S | (μmol/mol) | (μmol/mol) | Rate (%) |
| 0 min | 50 | 50 | 0.0 |
| 30 min | 49 | 39 | 20.4 |
| 60 min | 49 | 38 | 22.4 |
| 90 min | 49 | 38 | 22.4 |
| 120 min | 48 | 37 | 22.9 |

### EXAMPLE 2

After the microbe culture was boiled at 99° C. on a hot plate for 5 min and cooled to room temperature in the sterilizing step (S3) and then filtered (S4) after the culturing and mixing step (S2), it was tested for ability to remove ammonia (Table 5), trimethylamine (Table 6), formaldehyde (Table 7), and hydrogen sulfide (Table 8).

TABLE 5
Deodorizing Effect of Microbial Agent Prepared
by a Process Conducted to the Sterilizing (S3)
and the Purifying Step (S4) on Ammonia Gas

| | Test Result | | |
|---|---|---|---|
| Test Item | Blank Conc. | Sample Conc. | Deodorization |
| Ammonia NH₃ | (μmol/mol) | (μmol/mol) | Rate (%) |
| 0 min | 50 | 50 | 0.0 |
| 30 min | 49 | 2 | 95.9 |
| 60 min | 49 | 1 | 98.0 |
| 90 min | 49 | N.D | 100.0 |
| 120 min | 48 | N.D | 100.0 |

TABLE 6
Deodorizing Effect of Microbial Agent Prepared by
a Process Conducted to the Sterilizing (S3) and
the Purifying Step (S4) on Trimethylamine Gas

| Test Item | Test Result | | |
|---|---|---|---|
| Trimethylamine | Blank Conc. | Sample Conc. | Deodorization |
| (CH₃)₃N | (μmol/mol) | (μmol/mol) | Rate (%) |
| 0 min | 50 | 50 | 0.0 |
| 30 min | 49 | 2 | 95.9 |
| 60 min | 49 | 1 | 98.0 |
| 90 min | 49 | N.D | 100.0 |
| 120 min | 48 | N.D | 100.0 |

TABLE 7
Deodorizing Effect of Microbial Agent Prepared
by a Process Conducted to the Sterilizing (S3)
and the Purifying Step (S4) on Formaldehyde Gas

| Test Item | Test Result | | |
|---|---|---|---|
| Formaldehyde | Blank Conc. | Sample Conc. | Deodorization |
| HCHO | (μmol/mol) | (μmol/mol) | Rate (%) |
| 0 min | 50 | 50 | 0.0 |
| 30 min | 49 | 7 | 85.7 |
| 60 min | 49 | 6 | 87.8 |
| 90 min | 49 | 6 | 87.8 |
| 120 min | 48 | 5 | 89.6 |

TABLE 8
Deodorizing Effect of Microbial Agent Prepared by
a Process Conducted to the Sterilizing (S3) and
the Purifying Step (S4) on Hydrogen Sulfide Gas

| | |
|---|---|
| Test Item | Test Result |

| Hydrogen | Blank Conc. | Sample Conc. | Deodorization |
|---|---|---|---|
| Sulfide H₂S | (μmol/mol) | (μmol/mol) | Rate (%) |
| 0 min | 50 | 50 | 0.0 |
| 30 min | 49 | 30 | 38.8 |
| 60 min | 49 | 22 | 55.1 |
| 90 min | 49 | 18 | 63.3 |
| 120 min | 48 | 17 | 64.6 |

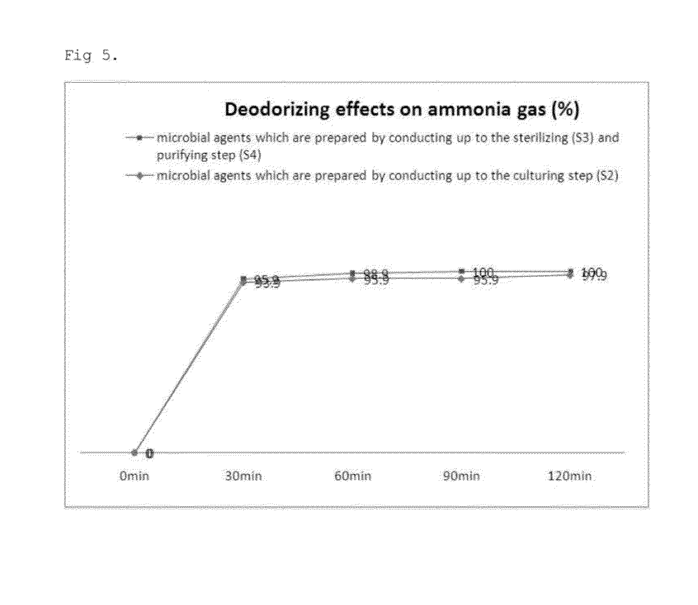

The test results are also depicted in FIGS. 5 to 8.

As can be seen in FIG. 5, both the microbial agents which were respectively prepared by conducting the process before the third step, and up to the third step in which the microbe culture was boiled at 99° C. on a hot plate for 5 min (S3), and the purifying step (S4) exhibited 98% or more deodorization of ammonia gas.

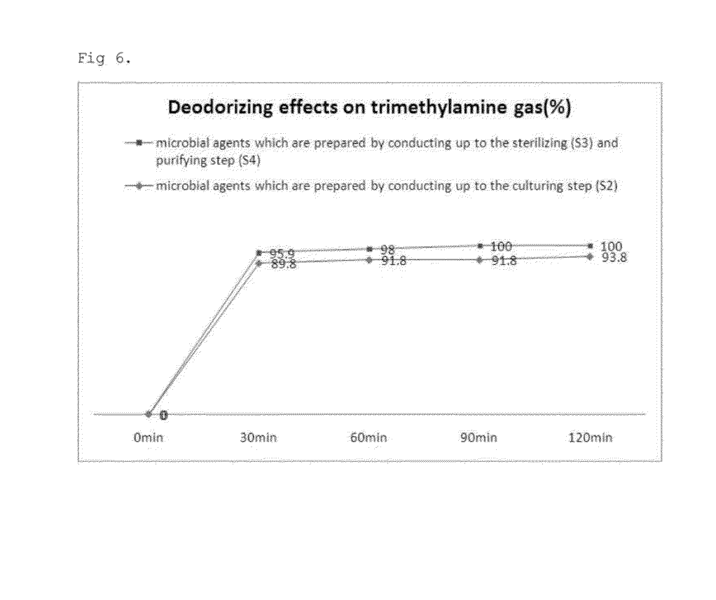

As can be seen in FIG. 6, the microbial agents which was prepared by conducting the process up to the third step in which the microbe culture was boiled at 99° C. on a hot plate for 5 min (S3), and the purifying step (S4) exhibited 99% or more deodorization of trimethylamine gas.

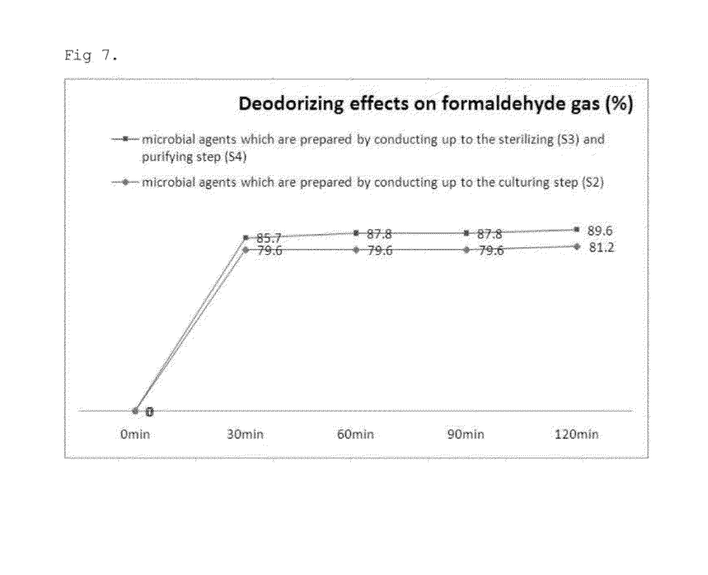

As can be seen in FIG. 7, both the microbial agents which were respectively prepared by conducting the process before the third step, and up to the third step in which the microbe culture was boiled at 99° C. on a hot plate for 5 min (S3) and the purifying step (S4) exhibited 98% or more deodorization of ammonia gas, with an improvement of the inventive microbial agent over the conventional microbial agent by 5-6%.

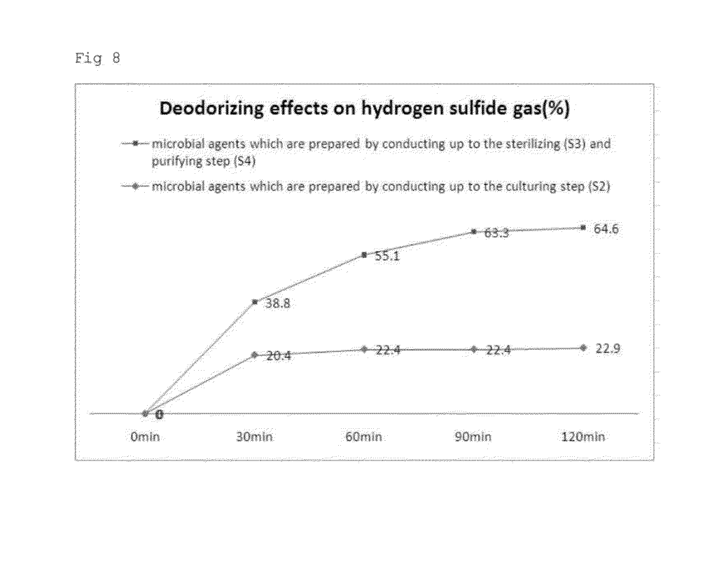

As can be seen in FIG. 8, the microbial agent which was prepared by conducting the process to the third step in which the microbe culture was boiled at 99° C. on a hot plate for 5 min (S3), and the purifying step (S4) was improved in the deodorization of ammonia gas by as great as 200%, compared to the microbial agent which was prepared by conducting the process prior to the third step.

As is apparent from FIG. 3, the microbe culture prepared following the sterilizing step (S3) and the purifying step (S4) is safe to fish. Also the microbial deodorizer of the present invention is demonstrated as being sate in terms of heavy metal and harmful ingredients, as indicated on the test report (Table 9) flow the Korea Conformity Laboratories.

TABLE 9
Test Report on Content of Heavy Metals and Harmful Components
in Microbial Deodorizer Prepared by Process Conducted to
the Sterilizing Step (S3) and Purifying Step (S4)

| Test | Report | Test | | |
|---|---|---|---|---|
| Item | No. | Method | Unit | Result |
| PAHS - | CT13-33902 | KS M 0027: | % | Not detected |
| Naphthalene | | 2008 | | (Detection |
| | | | | limit |
| | | | | 0.0005) |
| PAHS - | CT13-33902 | KS M 0027: | % | Not detected |
| Acenaphthylene | | 2008 | | (Detection |
| | | | | limit |
| | | | | 0.0005) |
| PAHS - | CT13-33902 | KS M 0027: | % | Not detected |
| Acenaphtent | | 2008 | | (Detection |
| | | | | limit |
| | | | | 0.0005) |
| PAHS - | CT13-33902 | KS M 0027: | % | Not detected |
| Fluorene | | 2008 | | (Detection |
| | | | | limit |
| | | | | 0.0005) |
| PAHS - | CT13-33902 | KS M 0027: | % | Not detected |
| Phenanthrene | | 2008 | | (Detection |
| | | | | limit |
| | | | | 0.0005) |
| PAHS - | CT13-33902 | KS M 0027: | % | Not detected |
| Anthracene | | 2008 | | (Detection |
| | | | | limit |
| | | | | 0.0005) |
| PAHS - | CT13-33902 | KS M 0027: | % | Not detected |
| Fluoranthrene | | 2008 | | (Detection |
| | | | | limit |
| | | | | 0.0005) |
| PAHS - | CT13-33902 | KS M 0027: | % | Not detected |
| Pyrene | | 2008 | | (Detection |
| | | | | Limit |
| | | | | 0.0005) |
| PAHS - | CT13-33902 | KS M 0027: | % | Not detected |
| Benzo (a) | | 2008 | | (Detection |
| fluoranthene | | | | limit |
| | | | | 0.0005) |
| PAHS - | CT13-33902 | KS M 0027: | % | Not detected |
| Chrysene | | 2008 | | (Detection |
| | | | | limit |
| | | | | 0.0005) |
| PAHS - | CT13-33902 | KS M 0027: | % | Not detected |
| Benzo (b) | | 2008 | | (Detection |
| fluoranthene | | | | limit |
| | | | | 0.0005) |
| PAHS - | CT13-33902 | KS M 0027: | % | Not detected |
| Benzo (k) | | 2008 | | (Detection |
| fluoranthene | | | | limit |
| | | | | 0.0005) |
| PAHS - | CT13-33902 | KS M 0027: | % | Not detected |
| Benzo [a] pyrene | | 2008 | | (Detection |
| | | | | limit |
| | | | | 0.0005) |
| PAHS - | CT13-33902 | KS M 0027: | % | Not detected |
| Dibenzo [a,h] | | 2008 | | (Detection |
| anthracene | | | | limit |
| | | | | 0.0005) |
| PAHS - | CT13-33902 | KS M 0027: | % | Not detected |
| Ideno [1,2,3-cd] | | 2008 | | (Detection |
| pyrene | | | | limit |
| | | | | 0.0005) |
| PAHS - | CT13-33902 | KS M 0027: | % | Not detected |
| Benzo [g,h,i] | | 2008 | | (Detection |
| perylene | | | | limit |
| | | | | 0.0005) |
| VACs - | CT13-33902 | KS M 0027: | % | Not detected |
| Benzene | | 2008 | | (Detection |
| | | | | limit |
| | | | | 0.0005) |
| VACs - | CT13-33902 | KS M 0027: | % | Not detected |
| Toluene | | 2008 | | (Detection |
| | | | | limit |
| | | | | 0.0005) |
| VACs - | CT13-33902 | KS M 0027: | % | Not detected |
| Ethylbenzene | | 2008 | | (Detection |
| | | | | limit |
| | | | | 0.0005) |
| VACs - | CT13-33902 | KS M 0027: | % | Not detected |
| Xylene | | 2008 | | (Detection |
| | | | | limit |
| | | | | 0.0005) |
| VACs - | CT13-33902 | KS M 0027: | % | Not detected |
| 1,4-dichloro- | | 2008 | | (Detection |
| benzene | | | | limit |
| | | | | 0.0005) |
| VACs - | CT13-33902 | KS M 0027: | % | Not detected |
| Styrene | | 2008 | | (Detection |
| | | | | limit |
| | | | | 0.0005) |
| Dichloro- | CT13-33902 | KS M 0027: | % | Not detected |
| methane | | 2008 | | (Detection |
| | | | | limit |
| | | | | 0.0005) |
| Chloroform | CT13-33902 | KS M 0027: | % | Not detected |
| | | 2008 | | (Detection |
| | | | | limit |
| | | | | 0.0005) |
| Carbon | CT13-33902 | KS M 0027: | % | Not detected |
| tetrachloride | | 2008 | | (Detection |
| | | | | limit |
| | | | | 0.0005) |
| 1,1,1-trichloro- | CT13-33902 | KS M 0027: | % | Not detected |
| ethylene | | 2008 | | (Detection |
| | | | | limit |
| | | | | 0.0005) |
| 1,1-dichloro- | CT13-33902 | KS M 0027: | % | Not detected |
| ethylene | | 2008 | | (Detection |
| | | | | limit |
| | | | | 0.0005) |
| Trichloro- | CT13-33902 | KS M 0027: | % | Not detected |
| ethylene | | 2008 | | (Detection |
| | | | | limit |
| | | | | 0.0005) |
| Tetrachloro- | CT13-33902 | KS M 0027: | % | Not detected |
| ethylene | | 2008 | | (Detection |
| | | | | limit |
| | | | | 0.0005) |
| Cd | CT13-33903 | KS M 0032: | mg/kg | Not detected |
| | | 2009 | | (Limit of |
| | | | | quantification |
| | | | | 0.02) |
| Cu | CT13-33903 | KS M 0032: | mg/kg | Not detected |
| | | 2009 | | (Limit of |
| | | | | quantification |
| | | | | 0.03) |
| Pb | CT13-33903 | KS M 0032: | mg/kg | Not detected |
| | | 2009 | | (Limit of |
| | | | | quantification |
| | | | | 0.20) |
| As | CT13-33903 | KS M 0032: | mg/kg | Not detected |
| | | 2009 | | (Limit of |
| | | | | quantification |
| | | | | 0.25) |
| Hg | CT13-33903 | KS M 0032: | mg/kg | Not detected |
| | | 2009 | | (Limit of |
| | | | | quantification |
| | | | | 0.01) |
| Cr | CT13-33903 | KS M 0032: | mg/kg | Not detected |
| | | 2009 | | (Limit of |
| | | | | quantification |
| | | | | 0.01) |
| Zn | CT13-33903 | KS M 0032: | mg/kg | Not detected |
| | | 2009 | | (Limit of |
| | | | | quantification |
| | | | | 0.07) |
| Ni | CT13-33903 | KS M 0032: | mg/kg | Not detected |
| | | 2009 | | (Limit of |
| | | | | quantification |
| | | | | 0.35) |

Prepared through a culturing step for amplifying, an autotrophic microbe of interest, and a sterilizing and purifying step for removing impurities and harmful microbes other than the autotrophic microbe, as described hitherto, the microbial deodorizer of the present invention is safe to the body and the environment as well as able to remove various odorous emissions from household sources in harmony with the virtuous cycle of the ecosystem. Thus, if does not work in an artificial or chemical manner, but in a natural manner, so as to recover the nature and environment.

The microbial deodorizer can be applied to various odorous sources including a bathtub, a toilet, a wardrobe, a refrigerator, a sink, a shoes cabinet, a drain, an automobile interior, a pet and its feces. Additionally, it can neutralize secondhand smoke, and odors associated with new furniture, freshly painted surfaces in offices, a sanatoriums, schools, public wash rooms, restaurants, hospitals, etc.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

I claim:

1. A method for preparing a microbial deodorizing agent, comprising:
a screening step (S1) of selecting an a autotrophic purple sulfur bacterial strain from the group consisting of Allochromatium palmeri, Ectothiorhodosinus mongolicus, Halochromatium roseum, and a combination thereof;
a culturing (S2-1) and mixing (S2-2) of the selected autotrophic microbe in a culture medium containing potassium phosphate, magnesium sulfate, sodium chloride, ammonium chloride, and calcium chloride, plus pure water, adjusted to a pH of 6.0-7.0, in an incubator at 43-45° C. for 190-210 hrs under light of 5000 Lux or higher until the number of the microbe reaches a density of 4-5×10⁵ cfu/ml or greater;
a sterilizing step (S3) of boiling the microbe culture at 99° C. for 5 min to remove possible impurities and other microbes;
a purifying step (S4) of cooling the boiled microbe culture to room temperature, stabilizing the microbe culture for 48-60 hrs without feeding an additional culture medium thereto, and filtering the microbe culture; and
a harvesting step (S5) of maintaining the autotrophic microbe filtrate at a density of 4-5×10⁵ cfu/ml.

2. A microbial deodorizer, using the microbial deodorizing agent prepared according to the method of claim 1, wherein the microbial deodorizer is harmless to a human body and the environment.
